(19) United States
(12) Patent Application Publication
(10) Pub. No.: US 2023/0277861 A1
(43) Pub. Date: Sep. 7, 2023

(54) STIMULATION ARRANGEMENTS, VENTILATION ARRANGEMENT, STIMULATION METHODS AND VENTILATION METHOD

(71) Applicant: STIMIT AG, Biel (CH)

(72) Inventor: Ronja MÜLLER-BRUHN, Windisch (CH)

(21) Appl. No.: 18/005,917

(22) PCT Filed: Jul. 22, 2021
(86) PCT No.: PCT/EP2021/070605

§ 371 Date: Jan. 18, 2023

(30) Foreign Application Priority Data
Jul. 22, 2020 (CH) 00911/20

(51) Int. Cl. A61N 2/00; A61N 2/02; A61M 16/00

(52) U.S. Cl. CPC A61N 2/002; A61N 2/006; A61N 2/02; A61M 16/0003; A61M 2016/0027; A61M 2205/057; A61M 2230/435; A61M 2016/0033; A61M 2230/432

(57) ABSTRACT

A stimulation arrangement comprising an induction device having a field generator configured to generate a spatial field, a sensor unit, and a control unit in communication with the induction device and the sensor unit. The field generator of the induction device is configured to be positioned at a human or animal patient such that an inspiration muscular structure of the patient is stimulatable by the spatial field, the sensor unit is configured to be positioned at the human or animal patient to sense a feedback of the respiratory system or the patient, and the control unit is configured to control the induction device to generate the spatial field and to receive a feedback signal from the sensor unit. The control unit is configured to evaluate the feedback signal received from the sensor unit, and to activate the field generator of the induction device when the feedback signal is indicative of an abnormality.

### TECHNICAL FIELD

The present invention relates to stimulation arrangement according to the preamble of the respective independent claims, to stimulation methods according to the preamble of the respective independent claims, to a ventilation arrangement according to the preamble of the respective independent claim and to a ventilation method according to the preamble of the respective independent claim.

### BACKGROUND ART

In the art it is known that electrical or electro-magnetic stimulation can be used for ventilating patients. Thereby, it is known to us such stimulation alone for ventilation or to assist conventional mechanical ventilation by stimulation. Moreover, various devices for and methods of ventilation by using electrical or electro-magnetic stimulation are described in the art. For example, WO 2020/079266 A1 describes such devices and methods.

However, there still is a need for further therapeutic applications and devices allowing such application in an efficient manner.

### DISCLOSURE OF THE INVENTION

According to the invention, needed improved arrangements and methods are suggested as explained below and as defined in the claims.

Accordingly, the present invention provides a stimulation arrangement comprising an induction device having a field generator configured to generate a spatial field, a sensor unit, and a control unit in communication with the induction device and the sensor unit, wherein the field generator of the induction device is configured to be positioned at a human or animal patient such that an inspiration muscular structure of the patient is stimulatable by the spatial field, the sensor unit is configured to be positioned at the human or animal patient to sense a feedback of the respiratory system or the patient, and the control unit is configured to control the induction device to generate the spatial field and to receive a feedback signal from the sensor unit. The control unit is configured to evaluate the feedback signal received from the sensor unit, and to activate the field generator of the induction device when the feedback signal is indicative of an abnormality.

The present invention also provides stimulation method of stimulating a patient to ventilate the patient or to assist breathing of the patient, comprising: positioning a field generator of an induction device at a human or animal patient, the field generator being configured to generate a spatial field, such that an inspiration muscular structure of the patient is stimulatable by the spatial field, positioning a sensor unit at the human or animal patient to sense a feedback of a respiratory system of the patient, evaluating a feedback signal provided by the sensor unit, and activating the field generator of the induction device such that the spatial field is generated when the feedback signal is indicative of an abnormality.

Unless being further specified in context of a concrete embodiment of one of the aspects of the invention, the following definitions and explanations do apply to all aspects of the invention described below:

For a control unit being in communication with any other component, it can be wiredly or wirelessly coupled to the other component. Like this, control signals can be transmitted to the other component for operating or controlling. Additionally or alternatively, signals such as sensor signals can be received by the control unit. For example, such sensor signals may represent a sensed dimension or physical property, e.g. for further evaluation.

The control unit can be any computing entity suitable for performing the tasks involved for controlling another component and/or for evaluating a signal such as sensed signal. It can be or comprise a laptop computer, a desktop computer, a server computer, a tablet, a smartphone or the like. The term “control unit” covers single devices as well as combined devices. The control unit can, for example, be a distributed system, such as a cloud solution, performing different tasks at different locations.

Typically, control units or computers involve a processor or central processing unit (CPU), a permanent data storage having a recording media such as a hard disk, a flash memory or the like, a random access memory (RAM), a read only memory (ROM), a communication adapter such as an universal serial bus (USB) adapter, a local area network (LAN) adapter, a wireless LAN (WLAN) adapter, a Bluetooth adapter or the like, and a physical user interface such as a keyboard, a mouse, a touch screen, a screen, a microphone, a speaker or the like. Control units or computers can be embodied with a broad variety of components.

The control unit can be partially or fully embodied as separate entity, or as a part integrated in any other device or entity. For example, it can be integrated in a ventilation arrangement such as being embodied in a ventilation machine used for ventilating a human or animal patient, and/or in the induction device.

The term “spatial field” as in context of the aspects of the invention described below relates to any field allowing stimulation of a target tissue of a patient. It may particularly involve an electric field or an electro-magnetic field. Such fields allow for directly stimulate muscular structures or indirectly stimulate muscular structures via the nervous system or via other muscular structures.

The term “pulses” in connection with the spatial field can relate to single pulses. Thereby, single pulses relate to the generation of the spatial field over a comparably short time and with a comparably long interruption between two subsequent pulses. Typically, single pulses are provided at frequencies lower than 10 Hertz (Hz) such as, e.g. at 5 Hz or below, or single pulses are initiated by the user or practitioner. The single pulses can have a temporal width of about 10 microseconds (μs) to about 300 μs. Such pulses can activate nerves and muscle structure and are identifiable by the patient or by a sensor. In particular, such single pulses may cause a single convulsion of a muscle or muscular structure.

In contrast thereto, when being generated as a train rather than in single pulses, the spatial field is either continuously generated or in sequences of pulses comparably quickly following each other. Such pulses can be provided in a frequency range of in between about 15 Hz and about 30 Hz. In particular, a train may achieve to activate a nerve or muscle such that a tetanic contraction or activation is induced. Advantageously, the train is provided by increasing the intensity (field strength) and/or frequency until a target intensity and frequency is achieved (ramp protocol). Like this, sudden convulsion or discomfort can be decreased. All of these parameters are summarized under the term “temporal characteristics” or “temporal parameters” of the spatial field. These temporal parameters can be adjusted manually via an input interface or be controlled automatically by an adjustment mechanism or control unit.

The parameters of the voltage or current waveform applied to generate the spatial field may affect the temporal characteristics of the spatial field, including pulse shape, amplitude, width, polarity, and repetition frequency; duration of and interval between bursts or trains of pulses; total number of pulses; and interval between stimulation sessions and total number of sessions have, amongst others, an influence on the field strength and determine if and with which intensity or “dose” a target area or target tissue can be activated.

The temporal characteristics and spatial distribution of the spatial field can be tuned in such a way that the desired activation (activation feedback) of the muscular structure is achieved. Thereby, the activation feedback (signal) may refer to a signal that indicates appropriate characteristics of muscular structure activation, e.g. a signal that reaches or exceeds a target value (threshold), a signal that exhibits a certain curve pattern or shape, a signal that fulfils a certain algorithm known to represent appropriate target muscular structure activation in the desired strength, or any combination thereof. The activation feedback (signal) may comprise a feedback in particular about a desired muscle activation strength that shall be reached before the adjustment mechanism stops variation. The appropriate activation feedback signal characteristics can for example be defined by a user via an input interface or be detected by algorithms.

The term “coil design” as used herein can be or comprise at least two coils or at least one cone shaped or otherwise curved or bulged coil, or at least one cylindrical or otherwise non-flat coil, or at least one small coil, i.e. a coil sufficiently small to generate a sharp electro-magnetic field such as a coil having a diameter of 3 cm or less. The targeted shape of the electro-magnetic field described herein can comprise a peak formed by the spatial electro-magnetic field. The electro-magnetic field generator can also be referred to as electro-magnetic field creator.

The targeted shape of the electro-magnetic field can be achieved by the electro-magnetic field being a locally constrained, targeted electric field, e.g., having a peak. It can be adapted to be active in a target area being the nerve area or tissue area that shall be activated with the electromagnetic-field (e.g. the phrenic nerve that shall be activated), which can be for example achieved by the peak in the electro-magnetic field (focality area). The targeted shape can generally be any shape of the electro-magnetic field or the time-dependent electric field component that allows to stimulate one or more target nerves effectively while minimizing other undesired co-stimulation effects of surrounding, above-lying or close-by tissues or nerves. A peak shape is such example, because it maximizes effects in a focality area and minimizes effects outside this area.

The parameters of the voltage or current waveform applied to the coil by a generator affect the temporal characteristics of the electromagnetic field, including pulse shape, amplitude, width, polarity, and repetition frequency; duration of and interval between bursts or trains of pulses; total number of pulses; and interval between stimulation sessions and total number of sessions have, amongst others, an influence on the field strength and determine if and with which intensity or “dose” a target area or target tissue can be activated.

The terms “positioned at” or “holding at” as used in connection with field generators of induction devices can relate to a field generator being physically in contact with a body of a patient or in close distance to it. The position and orientation of the field generator or a component of it can thereby be predefined or distinct to be appropriate for stimulating a target tissue. In order to be configured for being positioned at an appropriate location, a field generator can be formed to be suited to the location. Also, it can be equipped with an appropriate mounting structure for being secured at the location.

Similarly, the term “positioned at” in the context of a sensor unit can relate to a sensor unit or a part thereof being physically in contact with the body of the patient or distant to it. It may also involve being at least located inside the body such as, e.g., inside an oral cavity or the like. In order to be configured for being positioned at an appropriate location, a sensor unit can be formed to be suited to the location. Also, it can be equipped with an appropriate mounting structure for being secured at the location.

The term “sensor unit” can relate to a single sensor or a combination of sensors. A sensor unit can further include other means such as a communication adapter, a mounting arrangement or similar structures.

The term “abnormality” relates to an anomaly or an irregularity of the breathing of from the patient or from the respiratory system supporting the breath of the patient. The abnormality may occur for instance when the positive end-expiratory pressure of a respiratory system of the patient is below a certain value, or hyperventilation of the patient occurs, e.g. when the rate or tidal volume of breathing eliminates more carbon dioxide than the body can produce. The hyperventilation of the patient is often associated with lack of oxygen.

In order to evaluate the feedback signal if it is indicative of an abnormality, the present invention may provide the senor unit with a pressure sensor to sense a pressure of a respiratory system of the patient, and wherein the abnormality is when a positive end-expiratory pressure, PEEP, being below a predefined pressure threshold. In this case, if the PEEP measured by the pressure sensor is below predefined pressure threshold, which indicates an abnormality of the breathing, the field generator will be activated or adjusted, thereby starting or adjusting the stimulation in order to support the breathing of the patient.

In an exemplary embodiment, the sensor unit may comprise a hyperventilation sensor to sense a presence of hyperventilation of the patient, and wherein the abnormality is when a presence of hyperventilation of the patient is identified. In this case, if the hyperventilation of the patient occurs, which indicates an abnormality of the breathing, the field generator will be activated or adjusted, thereby starting or adjusting the stimulation in order to support the breathing of the patient.

In an exemplary embodiment, the sensor unit may comprise a sensor to sense an oxygen content of a gas supplied from the respiratory system to the patient, and wherein the abnormality is when the oxygen content is below a predefined oxygenation threshold. In this case, if the oxygenation of the air for the patent is too low, which indicates an abnormality of the breathing, the field generator will be activated or adjusted, thereby starting or adjusting the stimulation in order to support the breathing of the patient.

Hereinafter, several aspects and further preferred embodiments of the present invention will be described, where the abnormality refers to a low positive end-expiratory pressure, low oxygen content in the air, or occurrence of a hyperventilation.

In a first aspect, the invention is a stimulation arrangement comprising an induction device, a pressure sensor unit and a control unit. The induction device has a field generator configured to generate a spatial field. The control unit is in communication with the induction device and the pressure sensor.

The control unit can be any computing entity suitable for performing the tasks involved for controlling the induction device and for evaluating the pressure signal. The control unit can be partially or fully embodied as separate component, or as a component integrated in any other device or component. For example, it can be integrated in a ventilation arrangement such as being embodied in a ventilation machine used for ventilating the patient, and/or in the induction device.

The field generator of the induction device is configured to be positioned at a human or animal patient such that an inspiration muscular structure of the patient is stimulatable by the spatial field. Thereby, the stimulation of the inspiration muscular structure can be directly, indirectly such as via the nervous system, or a combination of the two. The pressure sensor unit is configured to be positioned at the human or animal patient to sense a pressure of a respiratory system of the patient.

The control unit is configured to control the induction device to generate the spatial field and to receive a pressure signal from the pressure sensor unit. Further, the control unit is configured to evaluate the pressure signal received from the pressure sensor unit to be indicative for a positive end-expiratory pressure and to activate the field generator of the induction device such that the spatial field is generated when the evaluated pressure signal is below a threshold. Thereby, the threshold can be a target pressure. Like this, the control unit can switch on the induction device or start application of the spatial field when threshold is reached.

The stimulation arrangement of the first aspect of the invention allows for correctly or appropriately setting a positive end-expiration pressure (PEEP). Thereby, a certain minimum pressure, e.g. the target pressure, can be securely kept inside a lung of the patient. Like this, alveolar collapse can be avoided or at least reduced. These effects may be especially important for patients with modified surfactant function such as lung disease patients. Also, the stimulation arrangement may allow for inducing deep inspirations resembling so-called sighs.

Thus, the stimulation arrangement of the first aspect of the invention allows for addressing one of the greatest challenges when mechanically ventilating patients, i.e. finding the correct setting for PEEP.

Preferably, the inspiration muscular structure comprises a diaphragm of the patient, an external intercostal muscle of the patient, an accessory muscle of inspiration of the patient, or a combination thereof. By stimulation any of these muscular structures or even a combination thereof, aspiration can efficiently be induced.

The pressure sensor unit preferably comprises an airway pressure sensor, and the pressure signal preferably has an airway pressure component. This allows for an efficient provision of a signal allowing for reliable setting of the PEEP.

Preferably, the pressure sensor unit comprises an esophageal pressure sensor and the pressure signal has an esophageal pressure component.

The control unit preferably is configured to evaluate the pressure signal by calculating a transpulmonary pressure by subtracting the esophageal pressure component of the pressure signal from the airway pressure component of the pressure signal. The transpulmonary pressure represents the true distending pressure of the lungs. Therefore, such embodiment allows for a particular beneficial setting of the PEEP.

Preferably, the control unit is configured to activate the induction device such that the field generator generates pulses of the spatial field. Thereby, the pulses of the spatial field preferably have a frequency of about 10 Hz to about 35 Hz. Such pulses of the spatial field allow for achieving an efficient stimulation or activation of the muscular structure to induce an inspiration.

In one preferred embodiment, the field generator of the induction device comprises an electrode and the spatial field generated by the field generator is an electric field. Such electrode allows for efficiently providing the spatial field. Alternatively, also other structure generating an electric field such as an antenna or the like can be used.

In another preferred embodiment, the field generator of the induction device comprises a coil design and the spatial field generated by the field generator is an electro-magnetic field having a targeted shape. Such coil design allows for generating the electro-magnetic field with the targeted shape, e.g., having a peak. Such shape allows for comparably precise stimulation or activation of a target tissue such as directly the inspiration muscular structure or any other structure connected to the inspiration muscular structure such as a tissue of the neural system or the like. Like this, stimulation of the inspiration muscular structure can be particularly efficient and convenient.

Preferably, the induction device comprises a second field generator configured to generate a second spatial field, the second field generator of the induction device is configured to be positioned at the human or animal patient such that an expiration muscular structure of the patient is stimulatable by the second spatial field, and the control unit is configured to operate the induction device such that the field generator and the second field generator generate coordinated pulses of the field and the second field to coordinately stimulate the inspiration muscular structure of the patient and the expiration muscular structure of the patient one after the other.

By having such second field generator, the inspiration as well as the expiration can be controlled by the stimulation arrangement. This allows for particular specific therapeutic applications. For example, the complete breathing process can be controlled and induced by the stimulation arrangement.

In one preferred embodiment, the second field generator of the induction device comprises a second electrode and the second spatial field generated by the field generator is a second electric field. Alternatively, also another structure generating an electric field such as an antenna or the like can be used.

In another preferred embodiment, the second field generator of the induction device comprises a second coil design and the second spatial field generated by the second field generator is a second electro-magnetic field.

The expiration muscular structure preferably comprises an abdominal muscle of the patient, an internal intercostal muscle of the patient, an accessory muscle of expiration of the patient, or a combination thereof.

In a particularly preferred embodiment, the second field generator of the induction device comprises the coil design such that it generates the second electro-magnetic field when being activated and the field generator of the induction device comprises the electrode such that it generates the electric field when being activated. Such combination of coil design and electrode allows for a sophisticated stimulation of the inspiration muscular structure and the expiration muscular structure. For example, the inspiration muscular structure can be activated by stimulating one or both Phrenic nerves, e.g., at the neck of the patient. Thereby, one or two coil designs can be positioned at the neck of the patient and the Phrenic nerve(s) can be stimulated by applying the targeted electro-magnetic field. This allows for a specific stimulation of the Phrenic nerve(s) and a limitation of stimulation of other tissue of the neck. The expiration muscular structure can, e.g., be activated by placing electrodes at the chest of the patient. This allows for directly stimulating the abdominal and/or internal intercostal muscles of the patient. As the electrode may generate a comparably wide spatial field the respective muscles can efficiently be stimulated. On a general note, stimulation of nerves or comparably small muscles may be more efficient or comfortable with a coils design. Conversely, stimulation of comparably large muscles may be more efficient or comfortable with electrodes.

Preferably, the control unit is configured to de-activate the second field generator of the induction device such that the second spatial field is not generated when the evaluated pressure signal is below the threshold. Like this, it can be achieved that the expiration is stopped before the pressure such as the transpulmonary or distending pressure is below a critical or desired value. In particular, the expiration can be stopped when transpulmonary pressure of 0 cm to 5 cm water column is reached at end expiration.

Preferably, the stimulation arrangement of the first aspect of the invention comprises an input structure configured to set the threshold. The input structure can be an interface for (semi-)automatic import of the threshold. More preferably, the input structure comprises a user interface such that a user or practitioner can actively set the threshold.

Preferably, the stimulation arrangement of the first aspect of the invention comprises a constraining device configured to provide an airflow resistance in the respiratory system of the patient. Like this, the expiration can be performed against a resistance which may result in a naturally created positive pressure in the lung. Like this, the alveoli can be kept open or they can be opened.

Preferably, the stimulation arrangement of the first aspect of the invention comprises an activator configured to manually activate the field generator of the induction device such that the spatial field is generated. Thereby, the activator preferably comprises a button accessible by the patient. Such embodiment allows for actively induce an inspiration. For example, if desired, it can be induced that a deep breath is performed.

Preferably, the field generator of the induction device is configured to be positioned at the patient such that a Phrenic nerve is in the spatial field generated by the field generator when the induction device is activated. Such embodiment allows for stimulating the Phrenic nerve which can efficiently activate the diaphragm such that inspiration occurs.

In a second aspect, the invention is a stimulation method of stimulating a patient to ventilate the patient or to assist breathing of the patient. The stimulation method of the second aspect comprises the steps of (i) positioning a field generator of an induction device at a human or animal patient, the field generator being configured to generate a spatial field, such that an inspiration muscular structure of the patient is stimulatable by the spatial field, (ii) positioning a pressure sensor unit at the human or animal patient to sense a pressure of a respiratory system of the patient, (iii) evaluating a pressure signal provided by the pressure sensor unit to be indicative for a positive end-expiratory pressure, and (iv) activating the field generator of the induction device such that the spatial field is generated when the evaluated pressure signal is below a threshold.

The stimulation method according to the second aspect of the invention and its preferred embodiments described in the following allow for efficiently achieving the effects and benefits of the stimulation arrangement of the first aspect of the invention and its preferred embodiments described above.

The inspiration muscular structure preferably comprises a diaphragm of the patient, an external intercostal muscle of the patient, an accessory muscle of inspiration of the patient, or a combination thereof.

Preferably, the pressure sensor unit comprises an airway pressure sensor, and the pressure signal has an airway pressure component.

Preferably, the pressure sensor unit comprises an esophageal pressure sensor and the pressure signal has an esophageal pressure component.

Preferably, evaluating the pressure signal provided by the pressure sensor unit comprises calculating a transpulmonary pressure by subtracting the esophageal pressure component of the pressure signal from the airway pressure component of the pressure signal.

Activating the field generator of the induction device preferably comprises generating pulses of the spatial field. Thereby, the pulses of the spatial field preferably have a frequency of about 10 Hz to about 35 Hz.

Preferably, the field generator of the induction device comprises an electrode and the spatial field generated by the field generator is an electric field. Alternatively, also another structure generating an electric field such as an antenna or the like can be used.

Preferably, the field generator of the induction device comprises a coil design and the spatial field generated by the field generator is an electro-magnetic field.

Preferably, the induction device comprises a second field generator configured to generate a second spatial field, and the second field generator of the induction device is configured to be positioned at the human or animal patient such that an expiration muscular structure of the patient is stimulatable by the second spatial field, wherein the induction device is operated such that the field generator and the second field generator generate coordinated pulses of the field and the second field to coordinately stimulate the inspiration muscular structure of the patient and the expiration muscular structure of the patient one after the other.

Thereby, the second field generator of the induction device preferably comprises a second electrode and the second spatial field generated by the field generator is a second electric field. Alternatively, also another structure generating an electric field such as an antenna or the like can be used.

Alternatively, the second field generator of the induction device preferably comprises a second coil design and the second spatial field generated by the second field generator is a second electro-magnetic field.

The expiration muscular structure preferably comprises an abdominal muscle of the patient, an internal intercostal muscle of the patient, an accessory muscle of expiration of the patient, or a combination thereof.

Preferably, the stimulation method of the second aspect of the invention comprises de-activating the second field generator of the induction device such that the second spatial field is not generated when the evaluated pressure signal is below the threshold.

Preferably, the stimulation method of the second aspect of the invention, comprising setting the threshold. Thereby, a user interface preferably is provided for setting the threshold.

Preferably, the stimulation method of the second aspect of the invention comprises providing an airflow resistance in the respiratory system of the patient.

Preferably, the stimulation method of the second aspect of the invention comprises manually activating the field generator of the induction device such that the spatial field is generated. Thereby, the field generator of the induction device preferably is activated by the patient or a practitioner pushing a button.

Preferably, the field generator of the induction device is configured to be positioned at the patient such that a Phrenic nerve is in the spatial field generated by the field generator when the induction device is activated.

In a third aspect, the invention is a ventilation arrangement comprising an induction device having a field generator configured to generate a spatial field, and a gas supply unit configured to be positioned at a human or animal patient to supply gas to a respiratory system of the patient. The gas supplied by the gas supply unit is air enriched with Oxygen. In the following the air enriched with Oxygen is also referred to as oxygenized air.

The gas supply unit can be configured to actively deliver or forward the oxygenized air to the respiratory system of the patient. Or, it can be configured to passively supply oxygenized air such that the respiratory system of the patient has to suck in the oxygenized air by itself. Further, the supply unit can be configured to allow active and passive supply or a combination thereof such that the oxygenized air is delivered at a certain pressure and, at the meantime, has to be sucked in by the patient. Also, it is possible that the gas supply unit provides the oxygenized air at a lower pressure such that the patient has to strongly suck in the oxygenized air.

The field generator of the induction device is configured to be positioned at a human or animal patient such that an inspiration muscular structure of the patient is stimulatable by the spatial field. Thereby, the stimulation of the inspiration muscular structure can be directly, indirectly such as via the nervous system, or a combination of the two.

Enriching the oxygen in the air results the air to have a higher oxygen concentration than regular air. Whereas regular air typically has between about 20.5% and 21.5% Oxygen, the enriched air in accordance with the invention has a higher Oxygen concentration. Preferably, the air enriched with Oxygen comprises more than about 21.5% Oxygen, more than about 22% Oxygen, more than about 22.5% Oxygen, or more than 23% Oxygen.

The ventilation arrangement of the third aspect of the invention allows for reducing or even cancelling mechanical ventilation of the patient. In particular, by applying the oxygenized air, less air volume has to be provided into the respiratory system of the patient for sufficient Oxygen consumption of the patient. Like this, the downsides of applying a comparably high pressure when ventilating patients can be reduced or eliminated. This may particularly be important or beneficial, when comparably vulnerable respiratory systems and specifically lungs are involved.

Thus, with the ventilation arrangement of the third aspect of the invention mechanical ventilation, e.g. by means of a breathing pump, or continuous positive airway pressure ventilation can be improved and provided more gently and efficient. Thereby, an appropriate Oxygen delivery to the patient can still be achieved or ensured.

Preferably, the ventilation arrangement of the third aspect of the invention comprises a control unit in communication with the induction device and the gas supply unit, wherein the control unit is configured to control the induction device to generate the spatial field and to control the gas supply unit to supply the air enriched with Oxygen to the respiratory system of the patient. Such control unit allows for an efficient (semi-)automatic application of many sophisticated ventilation therapies.

Preferably, the gas supply unit comprises an interface member configured to be arranged at the patient to allow supply of the air enriched with Oxygen to the respiratory system of the patient. Such interface member may be or comprise a mask such as a continuous positive airway pressure (CPAP) mask or a non-invasive mask, a tubus or the like. In particular, the interface member may be any structure known in conventional ventilation to be connected to the respiratory system of the patient, e.g. via the mouth and/or nose.

Preferably, the gas supply unit comprises a delivery member configured to generate a flow of gas to be delivered to the respiratory system of the patient. Such delivery member may involve a pump or a similar structure for forwarding the oxygenized air into the respiratory system of the patient.

Preferably, the inspiration muscular structure comprises a diaphragm of the patient, an external intercostal muscle of the patient, an accessory muscle of inspiration of the patient, or a combination thereof. By stimulation any of these muscular structures or even a combination thereof, aspiration can efficiently be induced.

Preferably, the control unit is configured to activate the induction device such that the field generator generates pulses of the spatial field. Thereby, the pulses of the spatial field preferably have a frequency of about 10 Hz to about 35 Hz. Such pulses of the spatial field allow for achieving an efficient stimulation or activation of the muscular structure to induce an inspiration.

In one preferred embodiment, the field generator of the induction device comprises an electrode and the spatial field generated by the field generator is an electric field. Such electrode allows for efficiently providing the spatial field. Alternatively, also another structure generating an electric field such as an antenna or the like can be used.

In another preferred embodiment, the field generator of the induction device comprises a coil design and the spatial field generated by the field generator is an electro-magnetic field. Such coil design allows for generating the electro-magnetic field with the targeted shape, e.g., having a peak. Such shape allows for comparably precise stimulation or activation of a target tissue such as directly the inspiration muscular structure or any other structure connected to the inspiration muscular structure such as a tissue of the neural system or the like. Like this, stimulation of the inspiration muscular structure can be particularly efficient and convenient.

Preferably, the induction device comprises a second field generator configured to generate a second spatial field, the second field generator of the induction device is configured to be positioned at the human or animal patient such that an expiration muscular structure of the patient is stimulatable by the second spatial field, and the control unit is configured to operate the induction device such that the field generator and the second field generator generate coordinated pulses of the field and the second field to coordinately stimulate the inspiration muscular structure of the patient and the expiration muscular structure of the patient one after the other.

By having such second field generator, the inspiration as well as the expiration can be controlled by the stimulation arrangement. This allows for particular specific therapeutic applications. For example, the complete breathing process can be controlled and induced by the stimulation arrangement.

In one preferred embodiment, the second field generator of the induction device comprises a second electrode and the second spatial field generated by the field generator is a second electric field. Alternatively, also another structure generating an electric field such as an antenna or the like can be used.

In another preferred embodiment, the second field generator of the induction device comprises a second coil design and the second spatial field generated by the second field generator is a second electro-magnetic field.

The expiration muscular structure comprises an abdominal muscle of the patient, an internal intercostal muscle of the patient, an accessory muscle of expiration of the patient, or a combination thereof.

In a particularly preferred embodiment, the second field generator of the induction device comprises the coil design such that it generates the second electro-magnetic field when being activated and the field generator of the induction device comprises the electrode such that it generates the electric field when being activated. Such combination of coil design and electrode allows for a sophisticated stimulation of the inspiration muscular structure and the expiration muscular structure. For example, the inspiration muscular structure can be activated by stimulating one or both Phrenic nerves, e.g., at the neck of the patient. Thereby, one or two coil designs can be positioned at the neck of the patient and the Phrenic nerve(s) can be stimulated by applying the targeted electro-magnetic field. This allows for a specific stimulation of the Phrenic nerve(s) and a limitation of stimulation of other tissue of the neck. The expiration muscular structure can, e.g., be activated by placing electrodes at the chest of the patient. This allows for directly stimulating the abdominal and/or internal intercostal muscles of the patient. As the electrode may generate a comparably wide spatial field the respective muscles can efficiently be stimulated. On a general note, stimulation of nerves or comparably small muscles may be more efficient or comfortable with a coils design. Conversely, stimulation of comparably large muscles may be more efficient or comfortable with electrodes.

Preferably, the ventilation arrangement of the third aspect of the invention comprises an activator configured to manually activate the field generator of the induction device such that the spatial field is generated. Thereby, the activator preferably comprises a button accessible by the patient. Such embodiment allows for actively induce an inspiration. For example, if desired, it can be induced that a deep breath is performed.

Preferably, the field generator of the induction device is configured to be positioned at the patient such that a Phrenic nerve is in the spatial field generated by the field generator when the induction device is activated.

In a fourth aspect, the invention is a ventilation method comprising: (i) positioning a field generator of an induction device at a human or animal patient, the field generator being configured to generate a spatial field, such that an inspiration muscular structure of the patient is stimulatable by the spatial field, and (ii) supplying gas to a respiratory system of the patient. The gas is air enriched with Oxygen, i.e. oxygenized air.

The ventilation method according to the fourth aspect of the invention and its preferred embodiments described in the following allow for efficiently achieving the effects and benefits of the ventilation arrangement of the third aspect of the invention and its preferred embodiments described above.

Preferably, the oxygenized air comprises more than about 21.5% Oxygen, more than about 22% Oxygen, more than about 22.5% Oxygen, or more than 23% Oxygen.

The ventilation method of the fourth aspect of the invention preferably comprises a step of arranging an interface member at the patient to allow supply of the oxygenized air to the respiratory system of the patient.

Preferably, the ventilation method of the fourth aspect of the invention comprises generating a flow of gas to be delivered to the respiratory system of the patient.

Preferably, the inspiration muscular structure comprises a diaphragm of the patient, an external intercostal muscle of the patient, an accessory muscle of inspiration of the patient, or a combination thereof.

Preferably, the ventilation method of the fourth aspect of the invention comprises activating the induction device such that the field generator generates pulses of the spatial field. Thereby, the pulses of the spatial field preferably have a frequency of about 10 Hz to about 35 Hz.

In one preferred embodiment, the field generator of the induction device comprises an electrode and the spatial field generated by the field generator is an electric field. Alternatively, also another structure generating an electric field such as an antenna or the like can be used.

In another preferred embodiment, the field generator of the induction device comprises a coil design and the spatial field generated by the field generator is an electro-magnetic field.

Preferably, the induction device comprises a second field generator configured to generate a second spatial field, and the second field generator of the induction device is configured to be positioned at the human or animal patient such that an expiration muscular structure of the patient is stimulatable by the second spatial field, wherein the induction device is operated such that the field generator and the second field generator generate coordinated pulses of the field and the second field to coordinately stimulate the inspiration muscular structure of the patient and the expiration muscular structure of the patient one after the other.

In one preferred embodiment, the second field generator of the induction device preferably comprises a second electrode and the second spatial field generated by the field generator is a second electric field. Alternatively, also another structure generating an electric field such as an antenna or the like can be used.

In another preferred embodiment, the second field generator of the induction device comprises a second coil design and the second spatial field generated by the second field generator is a second electro-magnetic field.

Preferably, the expiration muscular structure comprises an abdominal muscle of the patient, an internal intercostal muscle of the patient, an accessory muscle of expiration of the patient, or a combination thereof.

The ventilation method of the fourth aspect of the invention preferably comprises a step of manually activating the field generator of the induction device such that the spatial field is generated. Thereby, the field generator preferably is activated by the patient pushing a button.

Preferably, the field generator of the induction device is configured to be positioned at the patient such that a Phrenic nerve is in the spatial field generated by the field generator when the induction device is activated.

In a fifth aspect, the invention is a stimulation arrangement comprising an induction device having a field generator configured to generate a spatial field, a hyperventilation sensor unit and a control unit in communication with the induction device and the hyperventilation sensor unit. The field generator of the induction device is configured to be positioned at a human or animal patient such that an inspiration muscular structure of the patient is stimulatable by the spatial field. The hyperventilation sensor unit is configured to be positioned at the patient to sense an indicator representing hyperventilation of the patient. The control unit is configured to control the induction device to generate the spatial field. Further, the control unit is configured to receive an indicator signal from the hyperventilation sensor unit. The control unit is configured to evaluate the indicator signal received from the hyperventilation sensor unit to be indicative for hyperventilation of the patient and to activate the field generator of the induction device such that the spatial field is generated when the evaluated indicator signal represents hyperventilation.

By having the filed generator and the hyperventilation sensor, the stimulation of the fifth aspect of the invention allows for inducing or stimulating comparably deep inhalations. Like this, Oxygen levels in the blood of the patient can be staid in an appropriate range. Also, the active inspiration induction by means of the stimulation arrangement, hyperventilation, i.e. fast superfluous breathing attempts of the patient can be avoided.

In one preferred embodiment, the hyperventilation sensor unit is an airflow sensor configured to be arranged at the patient to sense an air flow in the respiratory system of the patient, the indicator signal is an airflow signal, and the control unit is configured such that the evaluated airflow signal represents hyperventilation when a breathing frequency determined from the airflow signal exceeds a threshold frequency. Thereby, the threshold frequency preferably is 15 per minute or more.

By observing the airflow, e.g. in the mouth of the patient, the breathing frequency can be appropriately monitored. Thus, such embodiment allows for efficiently identifying hyperventilation and to induce corrective measures by activating the inspiration muscular structure.

In another preferred embodiment, the hyperventilation sensor unit is a carbon dioxide sensor configured to be arranged at the patient to sense carbon dioxide levels in the air or blood of the patient, the indicator signal is a carbon dioxide signal, and the control unit is configured such that the evaluated carbon dioxide signal represents hyperventilation when a carbon dioxide level determined from the carbon dioxide signal deceeds a carbon dioxide threshold. Thereby, the carbon dioxide threshold preferably is 22 millimoles per liter (mmol/L) or less.

As an alternative or complement to observing airflow, the level of carbon dioxide in the blood or air(flow) can be observed. This allows for appropriately monitoring the breathing frequency. Thus, such embodiment also allows for efficiently identifying hyperventilation and to induce corrective measures by activating the inspiration muscular structure.

In a sixth aspect, the invention is a stimulation arrangement comprising an induction device having a field generator configured to generate a spatial field, an oxygenation sensor configured to be positioned at a human or animal patient to sense an oxygenation of the patient, and a control unit in communication with the induction device and the oxygenation sensor. The field generator of the induction device is configured to be positioned at the patient such that an inspiration muscular structure of the patient is stimulatable by the spatial field. The control unit is configured to control the induction device to generate the spatial field. The control unit is configured to receive an oxygenation signal from the oxygenation sensor unit. The control unit is configured to evaluate the oxygenation signal received from the oxygenation sensor unit and to activate the field generator of the induction device such that the spatial field is generated when the evaluated oxygenation signal is below an oxygenation threshold.

Similarly as for identifying hyperventilation, by having the filed generator and the oxygenation sensor, the stimulation of the sixth aspect of the invention allows for inducing or stimulating comparably deep inhalations if required, i.e. if the Oxygen level is too low. Like this, the Oxygen level in the blood of the patient can be staid in an appropriate range.

Preferably, in the stimulation arrangement of the fifth and sixth aspects of the invention the inspiration muscular structure comprises a diaphragm of the patient, an external intercostal muscle of the patient, an accessory muscle of inspiration of the patient, or a combination thereof.

Preferably, in the stimulation arrangement of the fifth and sixth aspects of the invention the control unit is configured to activate the induction device such that the field generator generates pulses of the spatial field. Thereby, the pulses of the spatial field have a frequency of about 10 Hz to about 35 Hz. Such pulses of the spatial field allow for achieving an efficient stimulation or activation of the muscular structure to induce an inspiration.

In one preferred embodiment of the stimulation arrangement of the fifth and sixth aspects of the invention, the field generator of the induction device comprises an electrode and the spatial field generated by the field generator is an electric field. Such electrode allows for efficiently providing the spatial field. Alternatively, also another structure generating an electric field such as an antenna or the like can be used.

In another preferred embodiment of the stimulation arrangement of the fifth and sixth aspects of the invention, the field generator of the induction device comprises a coil design and the spatial field generated by the field generator is an electro-magnetic field. Such electrode allows for efficiently providing the spatial field. Such coil design allows for generating the electro-magnetic field with the targeted shape, e.g., having a peak. Such shape allows for comparably precise stimulation or activation of a target tissue such as directly the inspiration muscular structure or any other structure connected to the inspiration muscular structure such as a tissue of the neural system or the like. Like this, stimulation of the inspiration muscular structure can be particularly efficient and convenient.

Preferably, in the stimulation arrangement of the fifth and sixth aspects of the invention the induction device comprises a second field generator configured to generate a second spatial field, the second field generator of the induction device is configured to be positioned at the human or animal patient such that an expiration muscular structure of the patient is stimulatable by the second spatial field, and the control unit is configured to operate the induction device such that the field generator and the second field generator generate coordinated pulses of the field and the second field to coordinately stimulate the inspiration muscular structure of the patient and the expiration muscular structure of the patient one after the other.

By having such second field generator, the inspiration as well as the expiration can be controlled by the stimulation arrangement. This allows for particular specific therapeutic applications. For example, the complete breathing process can be controlled and induced by the stimulation arrangement.

In one preferred embodiment of the stimulation arrangement of the fifth and sixth aspects of the invention, the second field generator of the induction device comprises a second electrode and the second spatial field generated by the field generator is a second electric field. Alternatively, also another structure generating an electric field such as an antenna or the like can be used.

In another preferred embodiment of the stimulation arrangement of the fifth and sixth aspects of the invention, the second field generator of the induction device comprises a second coil design and the second spatial field generated by the second field generator is a second electro-magnetic field.

Thereby, the expiration muscular structure preferably comprises an abdominal muscle of the patient, an internal intercostal muscle of the patient, an accessory muscle of expiration of the patient, or a combination thereof.

In a particularly preferred embodiment, the second field generator of the induction device comprises the coil design such that it generates the second electro-magnetic field when being activated and the field generator of the induction device comprises the electrode such that it generates the electric field when being activated. Such combination of coil design and electrode allows for a sophisticated stimulation of the inspiration muscular structure and the expiration muscular structure. For example, the inspiration muscular structure can be activated by stimulating one or both Phrenic nerves, e.g., at the neck of the patient. Thereby, one or two coil designs can be positioned at the neck of the patient and the Phrenic nerve(s) can be stimulated by applying the targeted electro-magnetic field. This allows for a specific stimulation of the Phrenic nerve(s) and a limitation of stimulation of other tissue of the neck. The expiration muscular structure can, e.g., be activated by placing electrodes at the chest of the patient. This allows for directly stimulating the abdominal and/or internal intercostal muscles of the patient. As the electrode may generate a comparably wide spatial field the respective muscles can efficiently be stimulated. On a general note, stimulation of nerves or comparably small muscles may be more efficient or comfortable with a coils design. Conversely, stimulation of comparably large muscles may be more efficient or comfortable with electrodes.

Preferably, the stimulation arrangement of the fifth and sixth aspects of the invention comprises an activator configured to manually activate the field generator of the induction device such that the spatial field is generated. Thereby, the activator comprises a button accessible by the patient. Such embodiment allows for actively induce an inspiration. For example, if desired, it can be induced that a deep breath is performed.

Preferably, in the stimulation arrangement of the fifth and sixth aspects of the invention the field generator of the induction device is configured to be positioned at the patient such that a Phrenic nerve is in the spatial field generated by the field generator when the induction device is activated.

In a seventh aspect, the invention is a stimulation method comprising (i) positioning a field generator of an induction device at a human or animal patient, the field generator being configured to generate a spatial field, such that an inspiration muscular structure of the patient is stimulatable by the spatial field, (ii) positioning a hyperventilation sensor unit at the patient to sense an indicator representing hyperventilation of the patient, (iii) evaluating an indicator signal received from the hyperventilation sensor unit to be indicative for hyperventilation of the patient, and (iv) activating the field generator of the induction device such that the spatial field is generated when the evaluated indicator signal represents hyperventilation.

The stimulation method according to the seventh aspect of the invention and its preferred embodiments described in the following allow for efficiently achieving the effects and benefits of the stimulation arrangement of the fifth aspect of the invention and its preferred embodiments described above.

Preferably, the hyperventilation sensor unit is an airflow sensor configured to be arranged at the patient to sense an air flow in the respiratory system of the patient, the indicator signal is an airflow signal, the evaluated airflow signal represents hyperventilation when a breathing frequency determined from the airflow signal exceeds a threshold frequency. Thereby, the threshold frequency preferably is 15 per minute or more.

Alternatively or complimentary, the hyperventilation sensor unit is a carbon dioxide sensor configured to be arranged at the patient to sense carbon dioxide levels in the air or blood of the patient, the indicator signal is a carbon dioxide signal, and the evaluated carbon dioxide signal represents hyperventilation when a carbon dioxide level determined from the carbon dioxide signal deceeds a carbon dioxide threshold. Thereby, the carbon dioxide threshold preferably is 22 mmol/L or less.

In an eight aspect, the invention is a stimulation method comprising (i) positioning a field generator of an induction device at a human or animal patient, the field generator being configured to generate a spatial field, such that an inspiration muscular structure of the patient is stimulatable by the spatial field, (ii) positioning an oxygenation sensor at a human or animal patient to sense an oxygenation of the patient, (iii) evaluating an oxygenation signal received from the oxygenation sensor unit, and (iv) activating the field generator of the induction device such that the spatial field is generated when the evaluated oxygenation signal is below an oxygenation threshold.

The stimulation method according to the eighth aspect of the invention and its preferred embodiments described in the following allow for efficiently achieving the effects and benefits of the stimulation arrangement of the sixth aspect of the invention and its preferred embodiments described above.

Preferably, the inspiration muscular structure comprises a diaphragm of the patient, an external intercostal muscle of the patient, an accessory muscle of inspiration of the patient, or a combination thereof.

Preferably, in the stimulation method of the seventh or eighth aspect of the invention the induction device is activated such that the field generator generates pulses of the spatial field. Thereby, the pulses of the spatial field preferably have a frequency of about 10 Hz to about 35 Hz.

In one preferred embodiment of the stimulation method of the seventh or eighth aspect of the invention, the field generator of the induction device comprises an electrode and the spatial field generated by the field generator is an electric field. Alternatively, also another structure generating an electric field such as an antenna or the like can be used.

In another preferred embodiment of the stimulation method of the seventh or eighth aspect of the invention, the field generator of the induction device comprises a coil design and the spatial field generated by the field generator is an electro-magnetic field.

Preferably, the stimulation method of the seventh or eighth aspect of the invention the induction device comprises a second field generator configured to generate a second spatial field, and the second field generator of the induction device is configured to be positioned at the human or animal patient such that an expiration muscular structure of the patient is stimulatable by the second spatial field, wherein the induction device is operated such that the field generator and the second field generator generate coordinated pulses of the field and the second field to coordinately stimulate the inspiration muscular structure of the patient and the expiration muscular structure of the patient one after the other.

In one preferred embodiment of the stimulation method of the seventh or eighth aspect of the invention, the second field generator of the induction device comprises a second electrode and the second spatial field generated by the field generator is a second electric field. Alternatively, also another structure generating an electric field such as an antenna or the like can be used.

In another preferred embodiment of the stimulation method of the seventh or eighth aspect of the invention, the second field generator of the induction device preferably comprises a second coil design and the second spatial field generated by the second field generator is a second electro-magnetic field.

Preferably, the expiration muscular structure comprises an abdominal muscle of the patient, an internal intercostal muscle of the patient, an accessory muscle of expiration of the patient, or a combination thereof.

Preferably, the stimulation method of the seventh or eighth aspect of the invention the induction device comprises a step of manually activating the field generator of the induction device such that the spatial field is generated. Thereby, the field generator preferably is activated by the patient pushing a button accessible.

Preferably, in the stimulation method of the seventh or eighth aspect of the invention the induction device the field generator of the induction device is configured to be positioned at the patient such that a Phrenic nerve is in the spatial field generated by the field generator when the induction device is activated.

### BRIEF DESCRIPTION OF THE DRAWINGS

The various aspects of the invention are described in more detail herein below by way of exemplary embodiments and with reference to the attached drawing.

FIG. 1 shows a schematic view of an embodiment of a stimulation and ventilation arrangement according to the invention implementing embodiments of methods according to the invention.

FIG. 1 shows exemplary stimulation arrangement 1 according to the invention. The ventilation machine 1 has a first implementation an electro-magnetic induction device 2 comprising an electro-magnetic field generator 21 with two coils 211 as coil design. The coils 211 are located in one common plane and configured to generate a spatial electro-magnetic field 212. When operated, the two coils 211 generate the electro-magnetic field 212 towards a neck 52 of a patient 5. The electro-magnetic field 212 has a central targeted shape with a target area 213 at which the electro-magnetic field 212 maximally extends into the neck 52. Alternatively, the induction device can comprise one or two electrodes for generate electrical fields for stimulate the nerve of the patient.

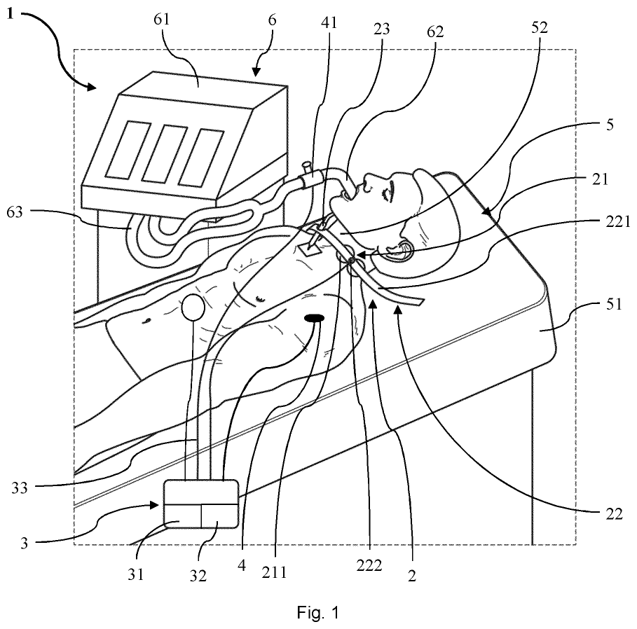

The induction device 2 has a mounting arrangement 22 with a neck arc 221 arranged at the neck 52 of the patient 5 and fixed to a bed 51 the patient 5 lies on. The neck arc 221 is equipped with a joint 222 as repositioning structure of an electro-magnetic field adjustment mechanism of the induction device 2. The joint 222 holds the coils 211 at the neck 52 of the patient 5.

The stimulation arrangement 1 further comprises a ventilator 11 as air flow generator from which ventilation tubes 13 extend. The ventilation machine 1 has a mouthpiece 12 as conduit interface of the ventilation machine 1 or as adapter of the EMI device. The mouthpiece 12 is applied to a mouth as entry point into the respiratory system of the patient 5. The ventilation tubes 13 are coupled to a flow sensor 41 of a sensor unit 4 or the induction device 2.

The stimulation arrangement 1 further has a controller 3 as a processing unit with a calibration unit 31 and a field adjustment unit 32 of the electro-magnetic field adjustment mechanism. The controller 3 is in communication with the flow sensor 41, the ventilator 11 and the joint 222 via respective wires 33.

The calibration unit 31 is configured to manipulate the joint 222 to automatically vary the position of the target area 213 of the electro-magnetic field 212 generated by the coils 211 and the controller 3 to vary the field strength of the electro-magnetic field 212. The aim of varying field strength and position of the electro-magnetic field 212 is to adjust the electro-magnetic field 212 such that it specifically stimulates a Phrenic nerve 53 of the patient 5. Upon stimulation of the Phrenic nerve 53, a diaphragm of the patient 5 is activated. Thereby, an airflow or breathing is induced which is sensed by the flow sensor 41.

The controller is configured to evaluate the feedback signal sent by the flow sensor 41, where the feedback signal includes the measurement airflow indicative of occurrence of hyperventilation, towards during the respiratory cycle, including the end of expiration. For instance, if the breathing frequency determined from the airflow exceeds a certain value, it indicates that the patient is under hyperventilation. In order to solve this abnormality, the stimulation can be activated, by generating the electro-magnetic field using the induction device 2.

Alternatively or additionally, the sensor unit 4 can includes a pressure sensor for measure the positive end-expiratory pressure, PEEP. If that is below a predefined value, it indicates that the breathing of the patient is abnormal. Hence, the the stimulation can be activated, by generating the electro-magnetic field using the induction device 2.

In other words, the field generator can receive an activation signal from the flow sensor 41 upon detection of detection of an abnormality. Further, it is configured to stop variation of the position of the target area 213 of the electro-magnetic field 212 and the controller 3 to stop variation of the field strength of the electro-magnetic field 212 when the activation feedback is received.

The ventilator 11 is configured to deliver air through the mouthpiece 12 into the respiratory system of the patient 5. Thereby, the controller 3 is configured to control the ventilator 11 to deliver air into the respiratory system according to a breathing scheme defined in the controller 3. In particular, the controller 3 regulates the activation of the diaphragm such that activation of the diaphragm via the Phrenic nerve 53 is coordinated with the ventilation of the patient 5.

As discussed above the sensor unit 4 can comprises a plurality of sensors, each being capable for detecting or measuring certain parameters related to the breathing and stimulation. For instance, the sensor unit 4 can comprise a flow sensor and a pressure sensor. In this case, the controller 3 can evaluate both the PEEP and the hyperventilation signals, and activate the stimulation or adjust the stimulation strength upon one of them or both of them being indicative of an abnormality.

In view of the above the present invention describes several conditions for starting and adjusting the stimulation. Each of the conditions indicate to an abnormality of the breathing. Upon detection of the abnormality, the stimulation can be controlled, e.g. starting the stimulation, or increase/decrease the intensity of the stimulation.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1-54. (canceled)

55. A stimulation arrangement comprising:
an induction device having a field generator configured to generate a spatial field;
a sensor unit; and
a control unit in communication with the induction device and the sensor unit, wherein
the field generator of the induction device is configured to be positioned at a human or animal patient such that an inspiration muscular structure of the patient is stimulable by the spatial field,
the sensor unit is configured to be positioned at the patient to sense a feedback from the patient or from a respiratory system of the patient,
the control unit is configured to control the induction device to generate the spatial field and to receive a feedback signal from the sensor unit, and
the control unit is configured to evaluate the feedback signal received from the sensor unit, and to activate the field generator of the induction device when the feedback signal is indicative of an abnormality.

56. The stimulation arrangement of claim 55, wherein the sensor unit comprises:
a pressure sensor unit to sense a pressure of a respiratory system of the patient, and wherein the abnormality is when a positive end-expiratory pressure, PEEP, is below a predefined pressure threshold, the pressure sensor unit including at least one of
an airway pressure sensor, and the feedback signal is a pressure signal having an airway pressure component, and
an esophageal pressure sensor and the pressure signal has an esophageal pressure component,
wherein the control unit is configured to evaluate the pressure signal by calculating a transpulmonary pressure by subtracting the esophageal pressure component of the pressure signal from the airway pressure component of the pressure signal; and/or
a sensor to sense an oxygen content of a gas supplied from the respiratory system to the patient, and wherein the abnormality is when the oxygen content is below a predefined oxygenation threshold.

57. The stimulation arrangement of claim 55, wherein the inspiration muscular structure comprises a diaphragm of the patient, an external intercostal muscle of the patient, an accessory muscle of inspiration of the patient, or a combination thereof.

58. The stimulation arrangement of claim 55, wherein the sensor unit comprises a hyperventilation sensor to sense a presence of hyperventilation of the patient, and the abnormality is when a presence of hyperventilation of the patient is identified, the hyperventilation sensor comprising at least one of
an airflow sensor configured to be arranged at the patient to sense an air flow in the respiratory system of the patient, wherein
the feedback signal is an airflow signal, and
the control unit is configured such that the evaluated airflow signal represents hyperventilation when a breathing frequency determined from the airflow signal exceeds a threshold frequency of 15 per minute or more; and/or
a carbon dioxide sensor configured to be arranged at the patient to sense carbon dioxide levels in the air or blood of the patient, wherein
the feedback signal is a carbon dioxide signal, and
the control unit is configured such that the evaluated carbon dioxide signal represents hyperventilation when a carbon dioxide level determined from the carbon dioxide signal is below a carbon dioxide threshold of 22 mmol/L or less.

59. The stimulation arrangement of claim 55, wherein the control unit is configured to activate the induction device such that the field generator generates pulses of the spatial field having a frequency of about 10 Hz to about 35 Hz.

60. The stimulation arrangement of claim 55,
wherein the field generator of the induction device comprises an electrode and the spatial field generated by the field generator is an electric field, or a coil design and the spatial field generated by the field generator is an electro-magnetic field; and/or
wherein the field generator of the induction device is configured to be positioned at the patient such that a Phrenic nerve is in the spatial field generated by the field generator when the induction device is activated.

61. The stimulation arrangement of claim 55, wherein
the induction device comprises a second field generator configured to generate a second spatial field,
the second field generator of the induction device is configured to be positioned at the patient such that an expiration muscular structure of the patient is stimulable by the second spatial field,
the control unit is configured to operate the induction device such that the field generator and the second field generator generate coordinated pulses of the spatial field and the second spatial field to coordinately stimulate the inspiration muscular structure of the patient and the expiration muscular structure of the patient one after the other,
the second field generator of the induction device comprises either
a second electrode and the second spatial field generated by the second field generator is a second electric field, or
a second coil design and the second spatial field generated by the second field generator is a second electro-magnetic field, and
the control unit is configured to de-activate the second field generator of the induction device such that the second spatial field is not generated when the feedback signal received from the sensor unit is indicative of the abnormality.

62. The stimulation arrangement of claim 56, comprising an input structure configured to set the predefined pressure threshold and/or the predefined oxygenation threshold, wherein the input structure comprises a user interface.

63. The stimulation arrangement of claim 55, further comprising:
a constraining device configured to provide an airflow resistance in the respiratory system of the patient; and
an activator configured to manually activate the field generator of the induction device such that the spatial field is generated, wherein the activator comprises a button accessible by the patient.

64. A stimulation method of stimulating a human or animal patient to ventilate the patient or to assist breathing of the patient, comprising:
positioning a field generator of an induction device at the patient, the field generator being configured to generate a spatial field, such that an inspiration muscular structure of the patient is stimulable by the spatial field;
positioning a sensor unit at the patient to sense a feedback from the patient or from a respiratory system of the patient;
evaluating a feedback signal provided by the sensor unit; and
activating the field generator of the induction device such that the spatial field is generated when the feedback signal is indicative of an abnormality.

65. The stimulation method of claim 64, wherein the inspiration muscular structure comprises a diaphragm of the patient, an external intercostal muscle of the patient, an accessory muscle of inspiration of the patient, or a combination thereof.

66. The stimulation method of claim 64, wherein the sensor unit comprises:
a pressure sensor unit to sense a pressure of the respiratory system of the patient, and wherein the abnormality is when a positive end-expiratory pressure, PEEP, is below a predefined pressure threshold, the pressure sensor unit including
an airway pressure sensor and the feedback signal is a pressure signal with an airway pressure component, and
an esophageal pressure sensor and the pressure signal has an esophageal pressure component, and
wherein evaluating the feedback signal includes evaluating the pressure signal provided by the pressure sensor unit and comprises calculating a transpulmonary pressure by subtracting the esophageal pressure component of the pressure signal from the airway pressure component of the pressure signal.

67. The stimulation method of claim 64, wherein the sensor unit comprises a hyperventilation sensor to sense a presence of hyperventilation of the patient, and wherein the abnormality is when a presence of hyperventilation of the patient is identified, the hyperventilation sensor unit.

68. The stimulation method of claim 67, wherein the hyperventilation sensor comprises at least one of:
an airflow sensor configured to be arranged at the patient to sense an air flow in the respiratory system of the patient, wherein
the feedback signal is an airflow signal, and
the evaluated airflow signal represents hyperventilation when a breathing frequency determined from the airflow signal exceeds a threshold frequency of 15 per minute or more; and/or
a carbon dioxide sensor configured to be arranged at the patient to sense carbon dioxide levels in the air or blood of the patient, wherein
the feedback signal is a carbon dioxide signal, and
the evaluated carbon dioxide signal represents hyperventilation when a carbon dioxide level determined from the carbon dioxide signal is below a carbon dioxide threshold of 22 mmol/L or less.

69. The stimulation method of claim 64, wherein the sensor unit comprises a sensor to sense an oxygen content of a gas supplied from the respiratory system to the patient, and wherein the abnormality is when the oxygen content is below a predefined oxygenation threshold.

70. The stimulation method of claim 64, wherein activating the field generator of the induction device comprises generating pulses of the spatial field, wherein the pulses of the spatial field have a frequency of about 10 Hz to about 35 Hz.

71. The stimulation method of claim 64, wherein the field generator of the induction device comprises:
an electrode and the spatial field generated by the field generator is an electric field; or
a coil design and the spatial field generated by the field generator is an electro-magnetic field.

72. The stimulation method of claim 64, wherein
the induction device comprises a second field generator configured to generate a second spatial field,
the second field generator of the induction device is configured to be positioned at the human or animal patient such that an expiration muscular structure of the patient is stimulable by the second spatial field,
the induction device is operated such that the field generator and the second field generator generate coordinated pulses of the spatial field and the second spatial field to coordinately stimulate the inspiration muscular structure of the patient and the expiration muscular structure of the patient one after the other, and
the second field generator of the induction device comprises a second electrode and the second spatial field generated by the field generator is a second electric field, or the second field generator of the induction device comprises a second coil design and the second spatial field generated by the second field generator is a second electro-magnetic field.

73. The stimulation method of claim 72, comprising de-activating the second field generator of the induction device such that the second spatial field is not generated when the feedback signal is indicative of the abnormality.

74. The stimulation method of claim 69, comprising setting the predefined pressure threshold and/or the predefined oxygenation threshold, wherein a user interface is provided for setting the predefined pressure threshold and/or the predefined oxygenation threshold.

75. The stimulation method of claim 64, comprising providing an airflow resistance in the respiratory system of the patient.

76. The stimulation method of claim 64, comprising manually activating the field generator of the induction device such that the spatial field is generated, wherein the field generator of the induction device is activated by the patient pushing a button.

77. The stimulation method of claim 64, wherein the field generator of the induction device is configured to be positioned at the patient such that a Phrenic nerve is in the spatial field generated by the field generator when the induction device is activated.
